(19) United States
(12) Patent Application Publication
(10) Pub. No.: US 2016/0152977 A1
(43) Pub. Date: Jun. 2, 2016

(54) ANALYZING METHOD FOR MICRO RNA ID AND BIOMARKERS RELATED TO COLON CANCER THROUGH THIS METHOD

(71) Applicant: JEONJU UNIVERSITY OFFICE OF INDUSTRY-UNIVERSITY COOPERATION, Jeonju-si (KR)

(72) Inventors: Jeong-Sang LEE, Jeonju-si (KR); Jae-Joong YUN, Jeonju-si (KR)

(21) Appl. No.: 14/954,806

(22) Filed: Nov. 30, 2015

(30) Foreign Application Priority Data
Dec. 1, 2014 (KR) 10-2014-0169452

(51) Int. Cl. C12N 15/113

(52) U.S. Cl. CPC C12N 15/113; C12N 2320/10; C12N 2310/141

(57) ABSTRACT

The present invention relates to an analysis method for the mi-RNA ID. More specifically, the invention relates to improve the analysis capabilities of the mi-RNA and to a method of analysis mi-RNA ID anticipating the generation of the cancer cells through this. In addition, the present invention is to apply to the biomarkers of colon cancer obtained by the mi-RNA ID analysis result gained through the analysis of the mi-RNA ID.

### CROSS-REFERENCE TO RELATED APPLICATION(S)

This application claims priority to and the benefit of Korean Patent Application No. 10-2014-0169452, filed on Dec. 1, 2014 in the Korean Intellectual Property Office, the entire content of which is incorporated herein by reference.

### DETAILED DESCRIPTION

1. Thecnical Field

The present invention relates to an analysis method for micro RNA (mi-RNA) ID. More specifically, the invention relates to improve the analysis capabilities of the mi-RNA for the acute analysis and to a method of analyzing mi-RNA ID anticipating the generation of cancer cells through this.

Furthermore, the invention relates to biomarkers discovered by the use of above method.

2. Background of Art

mi-RNA was first discovered by Victor Ambros and collaborators in 1993. While investigating the genes that control the timing of Caenorhabditis elegans laval development, they found that the synthesis of LIN-14 protein was affected by the short RNA fragment named lin-4. Since RNA fragment named let-7, act as a control factor in the same species, was additionally recognized, the presence and function of mi-RNAs were spotlighted.

mi-RNA, small single-stranded nucleotide consisting of 21 to 25 bases, is known to control the expression of various genes in eukaryotes. After the first mi-RNA expressed in C. elegance was identified in 1993, at present, more than 700 kinds of mi-RNA were found to be present in human cells.

The biosynthesis of mi-RNA mainly proceeds by two enzymes. First, the gene containing the mi-RNA is transcribed by RNA polymerase H or III to synthesize mi-RNA transcripts in a variety of sizes.

Pri-mi-RNA synthesized by this process has cap (7-methylguanylate cap) at 5′ tail and a poly-A at 3′ tail, respectively. The pri-mi-RNA is processed as a pre-mi-RNA that a nucleotide length of 70 by the microprocessor complex consisting of the ribonuclease enzyme called Drosha present in the nucleus and DGCR8 (DiGeorge critical region 8). The pre-mi-RNA comes to the cytoplasm through the Ran-GTP and exportin-5, then is processed into the mature mi-RNA duplexes consisting of 20-25 nucleotides by the second ribonuclease, Dicer, and TRBP (transactivating response RNA binding protein).

Among the two strands, one strand is decomposed, only the other strand combined with Ago (Argonaute) to constitute the RISC (RNA-induced silencing complex). TRBP regulates the expression of a target gene by inducing the binding of mi-RNA to Ago. In general, mi-RNA binds to 3′ untranslated region (3′UTR) to decrease the stability or the translation efficiency of mRNA resulting in the inhibition of a target gene expression.

While some mi-RNAs have its own promoter and transcription factors, most of the mi-RNAs are transcribed by the promoter and the transcription factor of the host gene including the mi-RNA. The transcription of mi-RNA is controlled by growth factors such as PDGF and TGF-3.

Binding to E-box, c-Myc induces the transcription of a mi-RNA-17-92 cluster, whose expression is increased in cancer cells. In addition, from the fact that six mi-RNAs, located in the mi-RNA-17-92 clusters, control the cell cycle. Therefore, mi-RNA is believed to be engaged in the mechanism that the over expression of c-Myc induces cancer. Besides the transcription factors, the expression of mi-RNA is also controlled by the epigenetic factors such as methylation and histone modification of DNA.

Recently, as mi-RNA was revealed to be a biologically important regulatory factor to control the expression of genes and 15,172 kinds of mi-RNA were identified among 32 species including animals and plants, bioinfomatic methods have been applied to process and analyze large amounts of data.

In order to store and manage the sequences, the information and the characteristics of mi-RNAs, several databases were set up. And miRBase, ASRP, micro RNA Map, etc. are reference database used widely. In addition, a variety of algorithms is developed to predict the candidates of mi-RNA gene or their target gene, as well as its applications.

As the methods for anticipating the candidates of mi-RNA genes, in general, RNA conformation-based search, homology 11 search for mi-RNAs with a similar sequences, and machine-learning approach, which mi-RNA characteristic value is applied in, are widely used.

The RNA conformation-based search is the method to use physical and chemical characteristics of the hairpin structure 12 in pre-mi-RNA. This method is the process of calculating whether a particular nucleotide sequence can have a thermodynamically stable hairpin structure for the anticipation of the candidates of mi-RNA genes.

The anticipation based on the homology search is the method of predicting candidates by calculating the probabilities of similarities in mi-RNA sequences, which is very useful to predict evolutionarily conserved mi-RNA sequences. The anticipation by the machine-learning method is a method widely used in the bioinformatics studies. This method is repeatedly training the machine with the characteristics of known mi-RNAs, such as nucleotide sequence, distribution, structural particularity, and evolutionary conserved features in order to predict the result in accordance with machine learning when a new sequence information is input.

As a prior art related to this, Korean Patent Publication No. 10-2013-0122541 (Nov. 7, 2013) discloses the method using the capillary electrophoresis system for detecting multiple mi-RNAs. However, this invention can not determine the exact characteristics of the mi-RNAs. Also, Korean Patent Publication No. 10-2014-0108913 (Sep. 15, 2014) relates to a method for quantitative analysis of mi-RNA. However, this invention is lack of the accuracy for the identification of an unknown mi-RNA. Furthermore, Korean Patent Publication No. 10-2014-0114684 (Sep. 29, 2014) relates to an automated mi-RNA search system. However, this invention only described the method for simple identification of mi-RNAs by using a mapping tool of reference mi-RNA database. With computation, Korean Patent Registration No. 10-0504039 (19 Aug. 2005) merely identifies whether the sequence is ncRNA.

The inventors have improved the capability of the mi-RNA analysis in order to solve the problems of the prior arts. As a result, efficient analysis of mi-RNA ID was achieved.

### PROBLEMS TO BE SOLVED BY EMBODIMENT OF THE INVENTION

The present invention improves the analysis capabilities of the mi-RNA for the acute analysis and provides a method of analyzing mi-RNA ID anticipating the generation of cancer cells through this.

Another object of the present invention is to apply the biomarker using the mi-RNA ID analysis result obtained through analysis of the mi-RNA ID.

### MEANS FOR SOLVING THE PROBLEMS

The invention is to provide an analysis method for mi-RNA ID, comprising (a) step of preparing an unknown biological sample;

(b) step of extracting the unknown mi-RNA from the above biological sample;
(c) step of obtaining common results from the mi-RNA extracted in the step (b) and a reference mi-RNA database;
(d) step of compensating the amount of mi-RNA obtained in the step (c) by the normalization for the comparison of mi-RNA results;
(e) step of performing the primary analysis of the mi-RNA results compensated by the normalization in the step (d), whose read count is more than 5, and the secondary analysis of the same results whose read count is between 2.5 and 5, by comparing to another database other than the above reference mi-RNA database;
(f) step of obtaining the mi-RNA ID from the results that are commonly derived from the results analyzed in the step (e).

On the other hand, other means of solving specific problems of the present invention are described in the detailed description of the invention.

### EFFECT OF THE EMBODIMENT OF THE INVENTION

Analysis method for mi-RNA ID according to the present invention is to improve the analysis capabilities of the mi-RNA which can be analyzed more accurately and also has an effect that can predict the generation of the common mi-RNAs through this method.

Furthermore, the present invention has the advantage able to provide mi-RNA biomarkers associated with colon carcinogenesis by using the analysis result of the common mi-RNA IDs obtained through the analysis of the mi-RNA IDs.

### BRIEF DESCRIPTION OF THE DRAWINGS

FIG. 1 is a flow diagram illustrating an analysis method for mi-RNA ID according to the present invention.

FIG. 2 is the result of mi-RNA clustering analysis of Oncogenes obtained from Example 1 (K-ras, PTEN, SMDA4, EGFR, PI3K).

FIG. 3 is the result of mi-RNA clustering analysis of Inflammatory-Genes obtained from the Example 2 (p65, p50, IKB-α, IKB-β, COX-2).

FIG. 4 is the result of mi-RNA clustering analysis of Cellular-Defense-Genes obtained from Example 3 (Keap1, Nrf2, HO-1).

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### MODE FOR CARRYING OUT EMBODIMENT OF THE INVENTION

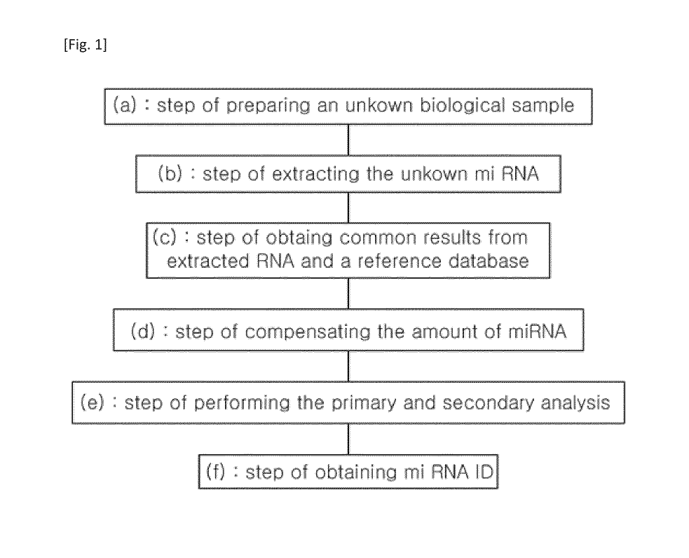

Hereinafter, with reference to FIGS. 1 to 4 the analysis of mi-RNA ID according to the present invention will be described in detail.

An analysis method for the mi-RNA ID according to the invention may comprise (a) step of preparing an unknown biological sample; (b) step of extracting the unknown mi-RNA from the above biological sample; (c) step of obtaining common results from the mi-RNA extracted in the step (b) and a reference mi-RNA database; (d) step of compensating the amount of mi-RNA obtained in the step (c) by the normalization for the comparison of mi-RNA results; (e) step of performing the primary analysis of the mi-RNA results compensated by the normalization in the step (d), whose read count is more than 5, and the secondary analysis of the same results whose read count is between 2.5 and 5, by comparing to another database other than the above reference mi-RNA database; (f) step of obtaining the mi-RNA ID from the results that are commonly derived from the results analyzed in the step (e). In embodiments according to the present invention, the term “read count” refers to a counted number of the sequence of a cluster that is obtained after the end of the RNA sequencing process which is ultimately the sequence of a section of a unique fragment, and may refer to the number of reads generated from a sequencing machine. In some embodiments, the term “read count” may refer to a counted number of base pairs (nucleotides). For example, the read count of 5 may refer to 5 matching RNA base pairs.

First, an analysis method for the mi-RNA ID is an initial step, and the unknown biological sample should be prepared. At this time, the unknown biological sample can be obtained from fresh or frozen colon cancer tissue, cell, blood, serum or plasma, however, are not limited.

Second, the present invention is to be of extracting total RNA including a mi-RNA from the unknown biological sample. The extraction method may utilize a variety of methods known in the art. It may preferably be extracted with trizol or triton X-100 as an extraction detergent.

Third, the present invention can obtain the common results from the mi-RNA extracted in the step (b) and a reference mi-RNA database. The reference mi-RNA database may utilize a variety of database known in the art. Preferably miRBase, ASRP, micro RNAMAP, miRGen, CoGemiR and miRZipTM can be used.

In the following, at the step of compensation, the amount of mi-RNA obtained in the step (c) is normalized for the comparison of the mi-RNA results.

Furthermore, in order to compared the mi-RNA result obtained in the step (d), the compensated mi-RNA result by the normalization is analyzed by the database other than the reference database used in the step (c). Perform the primary analysis of the result whose read count is more than 5, and do the secondary analysis of the same results whose read count is between 2.5 and 5. According to some example embodiments of the present invention, the term “normalization” refers to a process in which data attributes within a data model are organized to increase the cohesion of entity types. In other words, the goal of data normalization may be to reduce and even eliminate data redundancy, and important consideration for application developers because it is very difficult to store objects in a relational database that maintains the same information in several places.

Oncogenes and tumor suppressor genes of the reference database are K-ras, TGF-β, TGF-BR2, Smads4, PTEN, PI3K, EGFR, VEGF, MYC, p53, APC, FOXO1m Braf, COX-2, HO-1, Sirt-1, and the like. Also, the comparison results may be obtained through the combination of at least one or more tumor suppressor genes of the reference database.

The K-ras gene, first discovered in 1967, is one of the genes involved in the generation of colon cancer, and comprising EGFR cell signaling pathway.

In addition, TGF-β and TGF-BR2 strongly inhibit the proliferation of immune cells or epithelial cells. When normal cells convert to cancer cells, their inhibition of cell proliferation become lost.

Smads are reported to transduce TGF signaling between the nucleus and cytoplasm, and phosphorylated by the TGF-I receptor for the activation. Smad-4 is known as an important factor of the generation and progression of the tumor. In practice, several reports observed the mutagenic changes in the tumor cells by Smad-4.

PTEN gene is known to play a key role in preventing the generation and progression of several types of cancer. Many studies until now have indicated when PTEN is lost its function or mutated, malignant cells repeatedly proliferate without control to result in the development of cancer.

Activated PI3K pathway at the cell surface results in the occurrence of cancer.

Vascular Endothelial Growth Factor (VEGF) plays a role to induce angiogenesis and to the permeability of blood vessels. The interaction and adhesion between cells are inevitable for these roles.

MYC gene over-expression is reported to be related to the conversion of normal cells to tumor.

APC gene is composed of 15 exons and produces the protein consisting of a total of 2843 amino acids, which regulates cell growth through the signal transduction of β-catenin involved in the adhesion between cells.

p53 is a tumor suppressor protein and a human p53 is encrypted with the TP53 gene. p53 is very important in the prevention of cancer as a cell cycle inhibitor of multi cellular organisms.

Cyclooxygenase-2 (COX-2) is generally known to be over expressed in the tumor tissue. The COX-2 overexpression is often to inhibit the apoptosis of tumor cells and promote cell division.

SIRT-1 involves in gene expression, glucose metabolism, insulin production, inflammatory response and nerve cell protection by controlling the development, aging and death of cells. In addition, it is involved in the occurrence of the variety of geriatric diseases, such as cancer, metabolic diseases, obesity, inflammatory diseases, diabetes, heart disease and degenerative brain diseases.

Heamoxygenase-1 (HO-1) is induced by ultraviolet radiation, hydrogen peroxide, cytokine, hypoxia and the glutathione(GSH) consumption. This can be thought as a cellular defense mechanism against the stress. Carbon monoxide (CO), a reaction product, suppresses the inflammatory factor. Modifying the structure of enzymes having heme or other metal ions, it causes the change of their activities and inhibits the apoptosis.

HO-1 gene expression is controlled by a variety of transcription factors. As a representative, NF-E2-related factor-2 (Nrf2) can be mentioned. Nrf2, as a redox-sensitive transcription factor, controls the expression of a variety of antioxidant enzymes. Nrf2 normally forms an inactive complex with Keap1 in the cytoplasm. However, in the activated condition, it moves to the nucleus to combine with antioxidant response element (ARE) for increasing the expression of various types of detoxification and antioxidant enzymes, such as NAD(P) H: quinone oxidoreductase( NQO1), glutathione-S-transferase GST), gamma-glutamate cystein ligase (GCL), HO-1, and the like. The treatment of tetrahydropapaveroline (THP) to PC12 cells activates Nrf2. Then, Nrf2 is translocated and combined with ARE binding cites for the expression of antioxidant enzymes, such as HO-1, resulting in the cell protection effect.

In order to use other database than the reference mi-RNA database, for the analysis, miRWalK, miRanda, miRDB, RNA-22, Targetscan, TarBase, miRecords, MiRscan, ProMiR, miRDeep, miRanalyzer, PicTar, DIANA microT, RNAhybrid, Mir Target2 and the like can be used. Also, at least one or more different databases can be used for the analysis. It is possible to obtain the mi-RNA ID of an unknown biological sample through the analysis of the different databases.

Meanwhile, when using the above other databases, the read count between 0.1 and <20 can be used. If the read count is greater than 20, there is a problem in accuracy. If the read count is less than 0.1, it is inefficient for consuming plenty of time in the analysis. Therefore, the read count should be in the range between 0.1 and 20, and also it is preferable to analyze the read count twice. More preferably, perform the primary analysis with the read count more than 5, and secondly set the range of the read count between 2.5 and 5 for the secondary analysis.

Indeed, the results obtained through the analysis of the mi-RNA ID can be used for the biomarker.

### EXPERIMENTAL EXAMPLE 1

### RNA Extraction Method from the Frozen Colon Cancer Tissue

First, a biological sample of frozen colon cancer tissue was prepared. With trizol, from the prepared biological sample, total RNA was extracted. The extraction method was as follows; 50-100 mg of the bio sample was finely crushed and placed in trizol 1 ml. Then 0.2 ml of chloroform was added. After 3 minutes at room temperature, it was centrifuged for 15 minutes at 12000 rpm. The supernatant was transferred to a new tube and 0.5 ml of isopropyl alcohol was added. After 10 minutes, it was centrifuged for 10 minutes at 12000 rpm and the supernatant was discarded. Followed by the addition of DEPC treated 75% ethanol 1 ml to the RNA pellet. After taping, it was centrifuged for 5 minutes at 12000 rpm with special column for collecting small RNAs. Subsequently the supernatant was again discarded, and the remaining RNA pellet was dried for 10 minutes at room temperature.

### EXPERIMENTAL EXAMPLE 2

### RNA Extraction Method of Living Cells

A sample of the biological living cells was prepared and the RNA was extracted from the sample with Trizol solution. The extraction method is the same as the Experimental Example 1.

### EXPERIMENTAL EXAMPLE 3

### RNA Extraction Method of Somatic Cells

A sample of the somatic cells was prepared, and the RNA was extracted from the sample with Trizol solution. The extraction method is the same as the Experimental Example 1.

### EXAMPLE 1

The RNA pellet prepared in Experimental Example 1 was used, and the base sequence of RNA was analyzed by sequencing systems. The information of analyzed base sequence of RNA is identified with the reference mi-RNA database. A table of normalized RNA information was sorted. The above table is used to compensate the reference amount, and the normalized RNA result was identified with other databases: miRWalK, miRanda, miRDB, RNA-22, Targetscan and TarBase, except the reference mi-RNA database. mi-RNA ID from this analysis was identified.

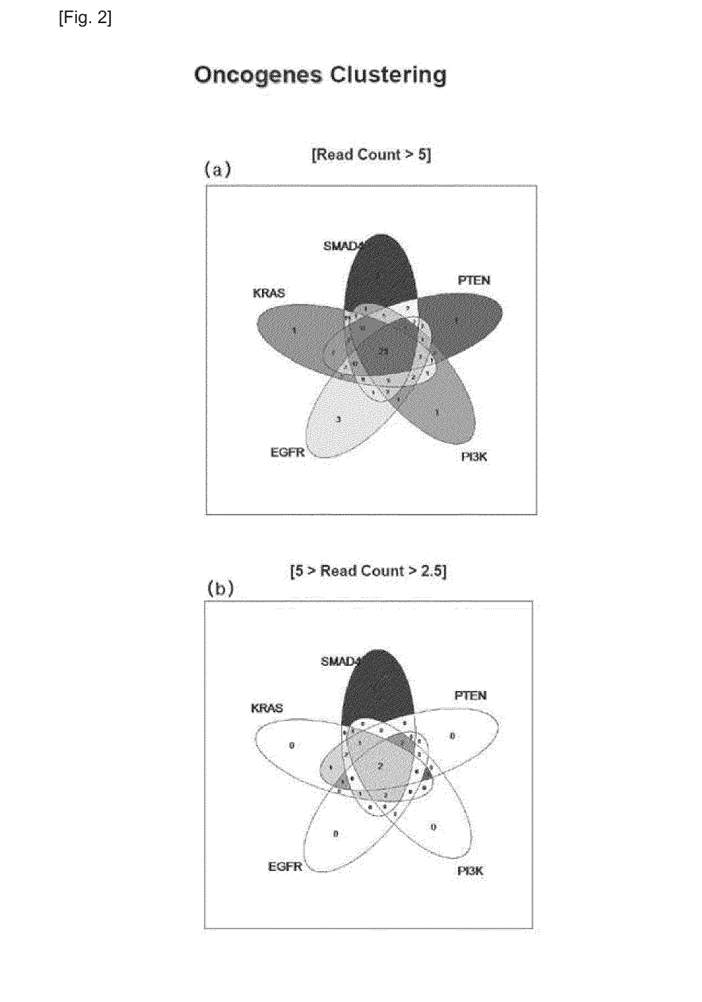

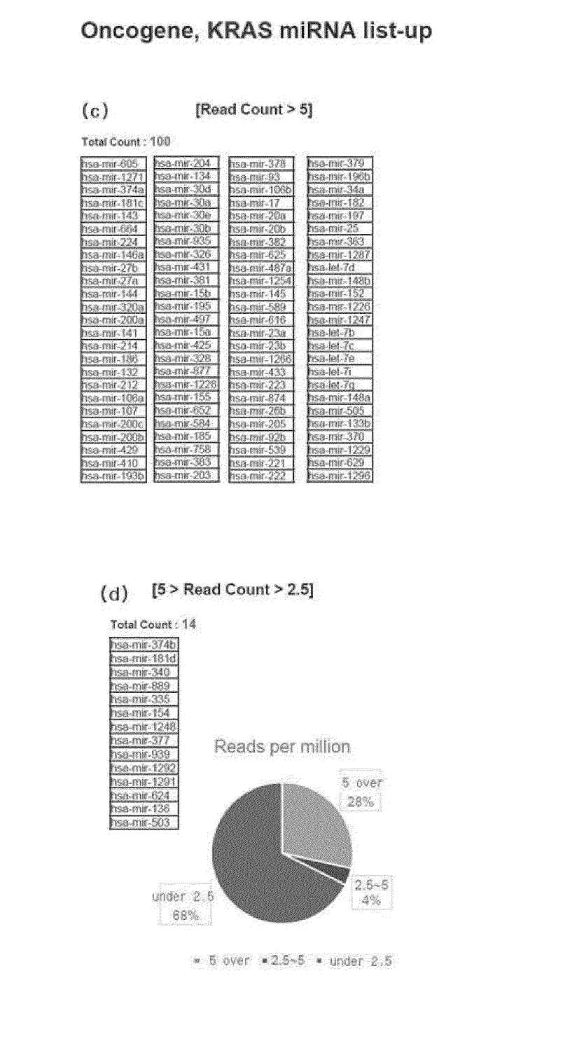

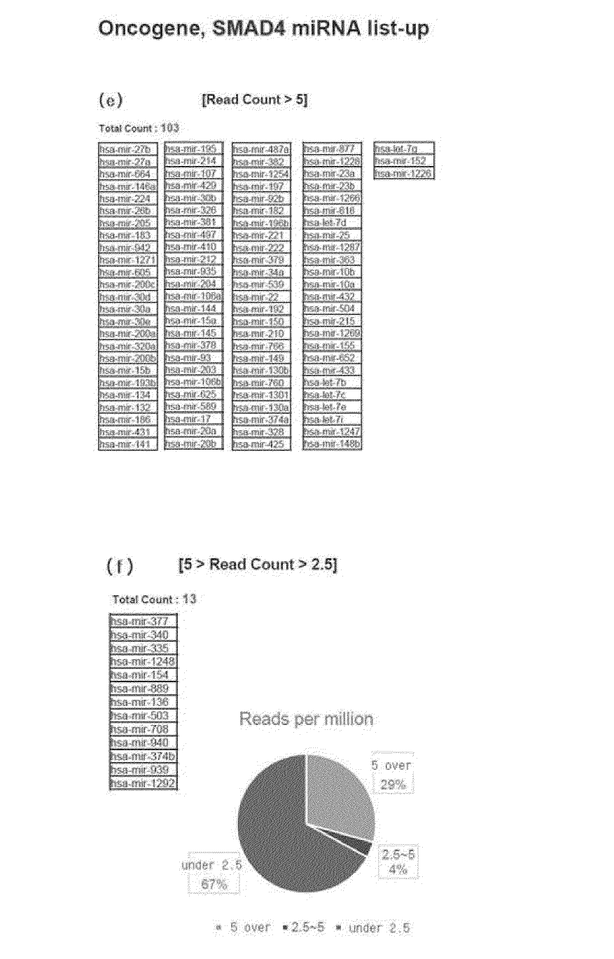

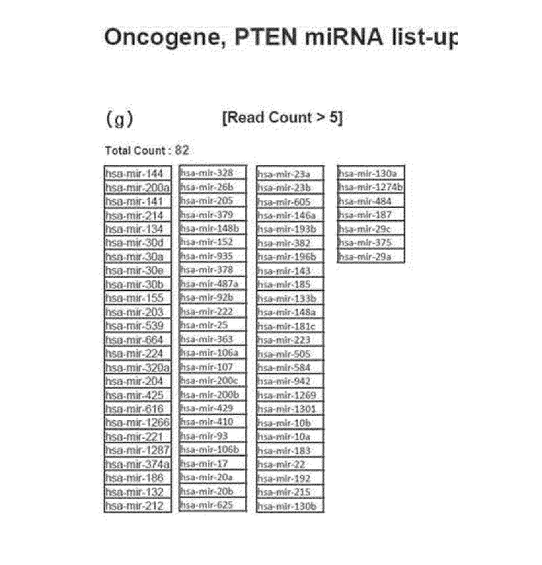

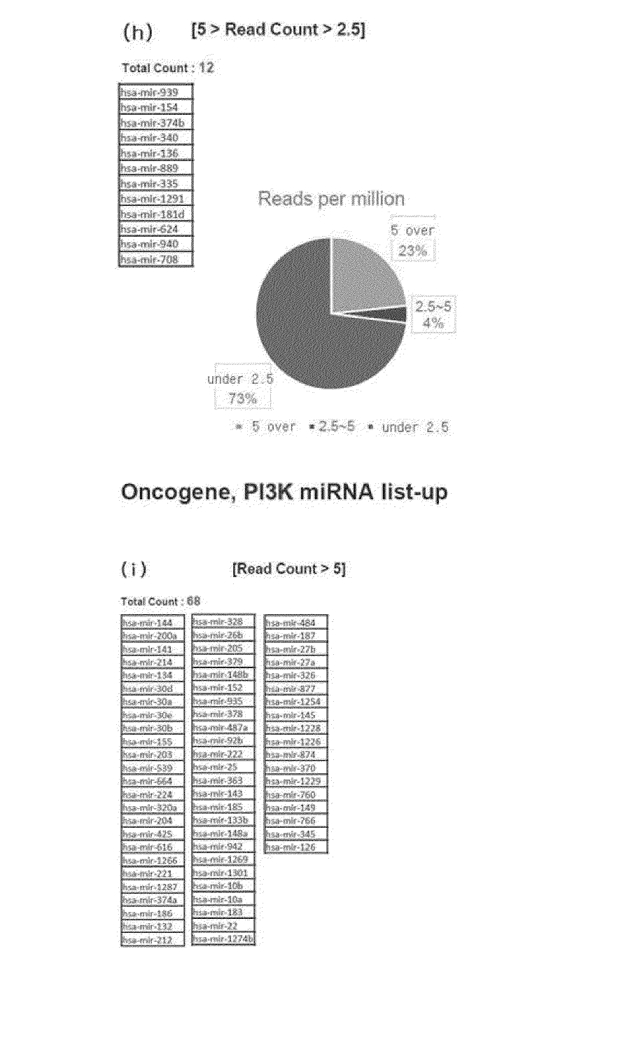

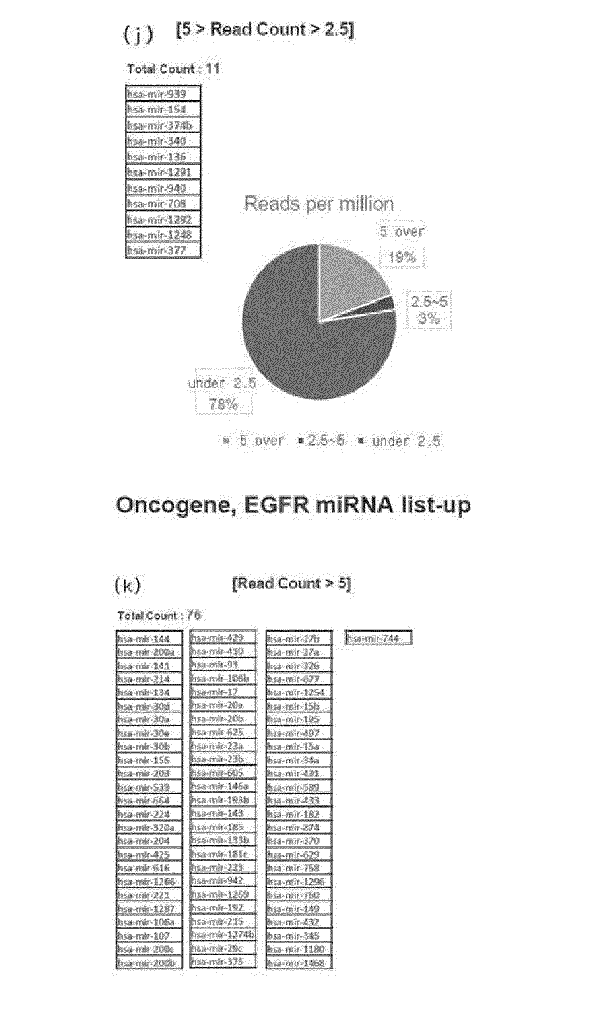

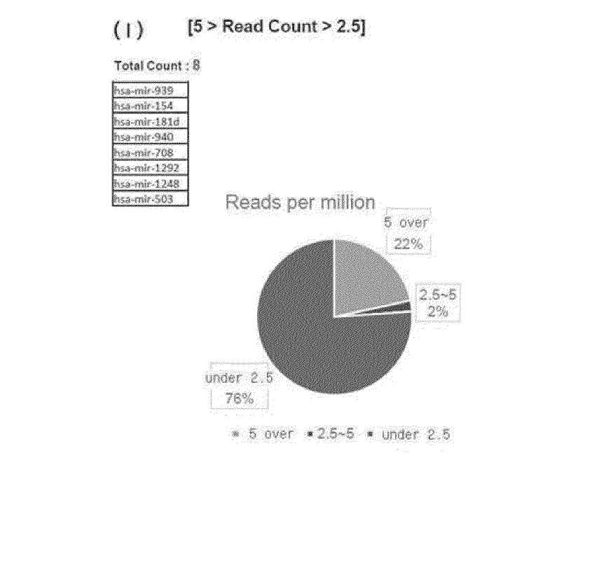

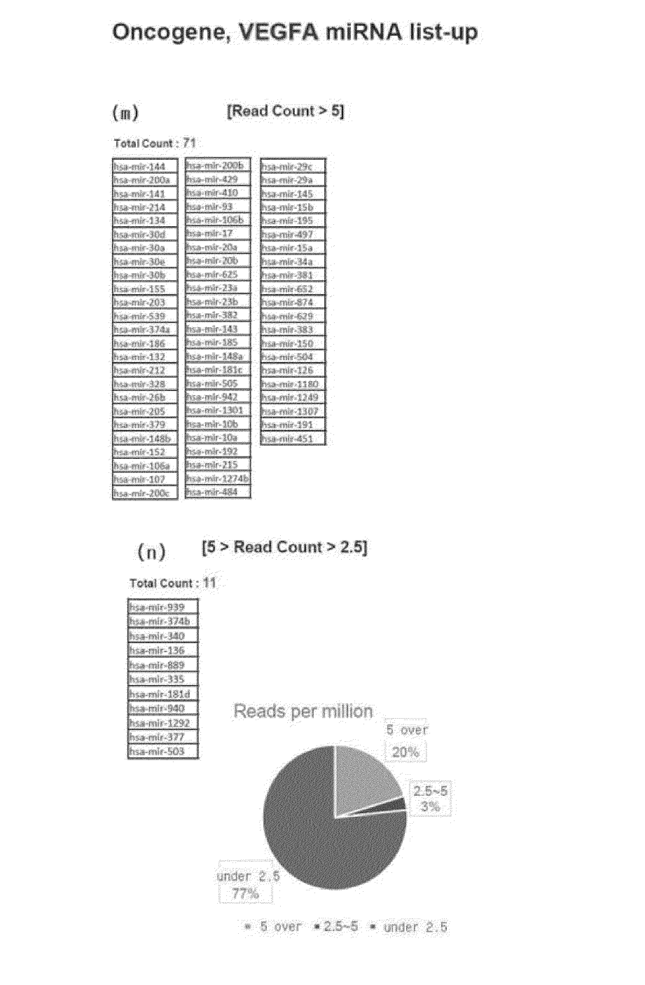

With miRWalK, miRanda, miRDB, RNA-22, Targetscan and TarBase D/B, the above normalized RNA result is analyzed to predict its target and compare the gene with K-ras, PTEN, SMDA4, EGFR and PI3K. Genes whose read count for each oncogene was at least 5 were obtained. Also the genes having the read count between 2.5 and 5 were gained. The results of oncogenes clustering analysis results are shown in FIG. 2, and thus mi-RNA ID was identified through this oncogene clustering analysis.

### EXAMPLE 2

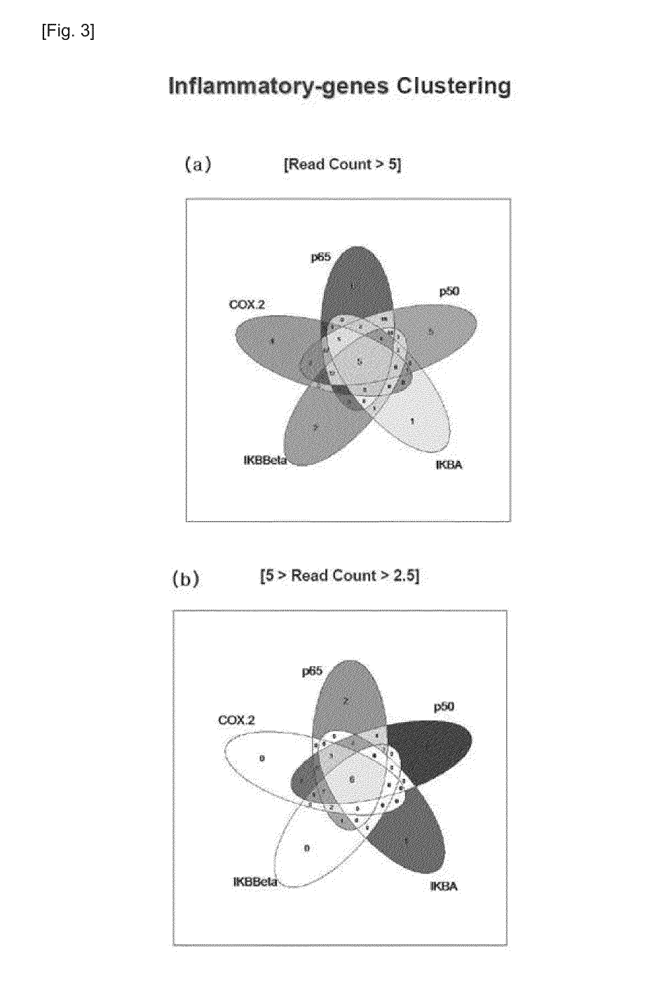

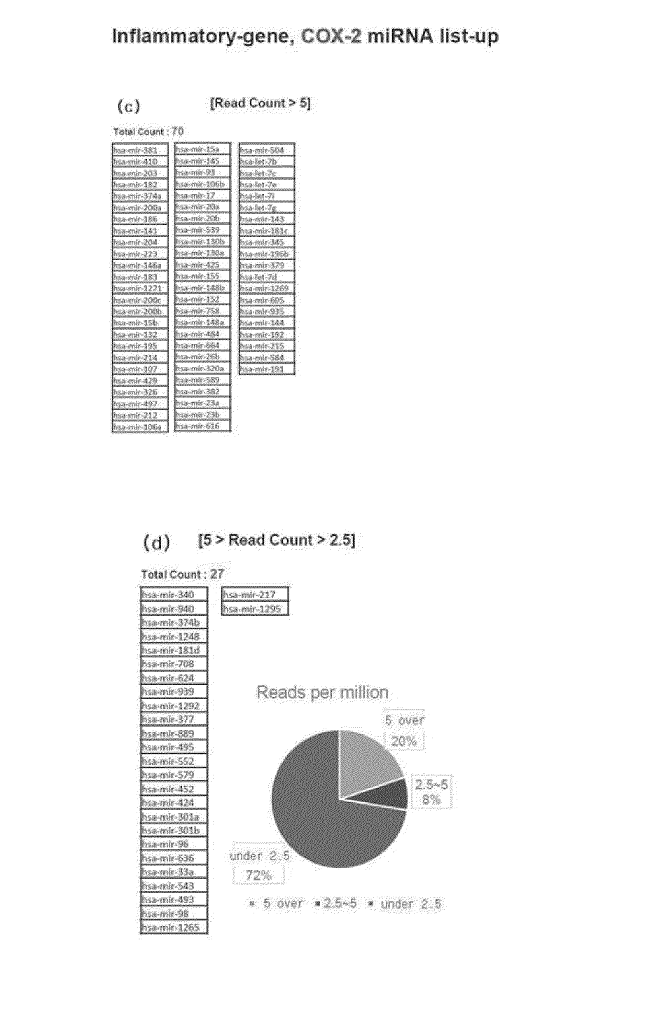

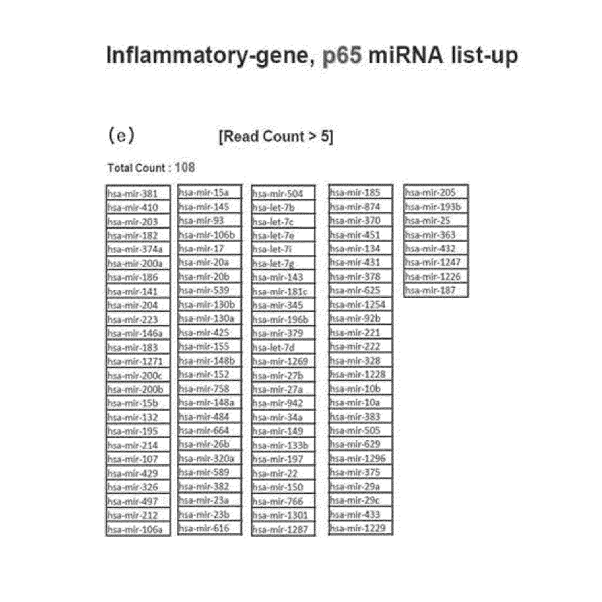

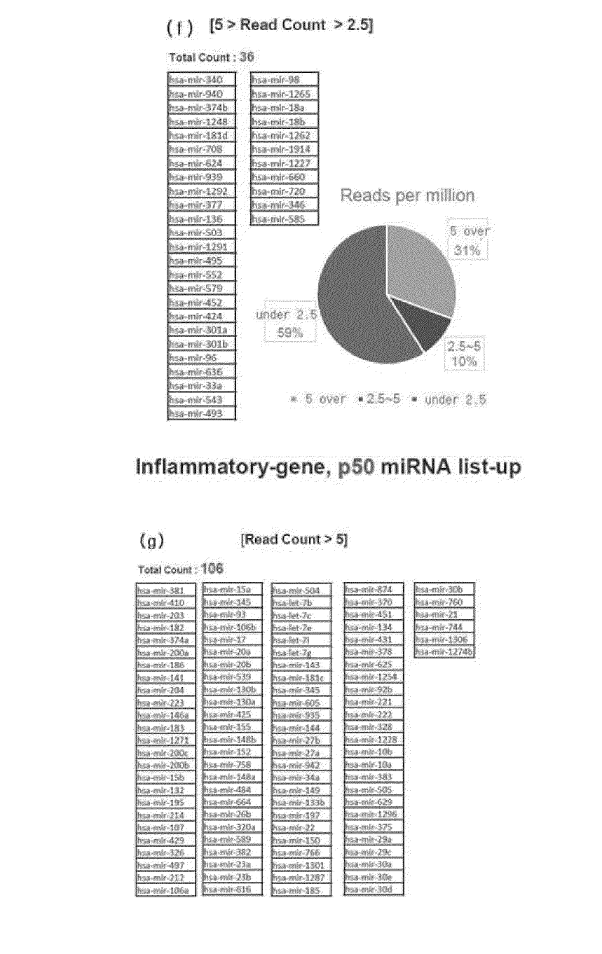

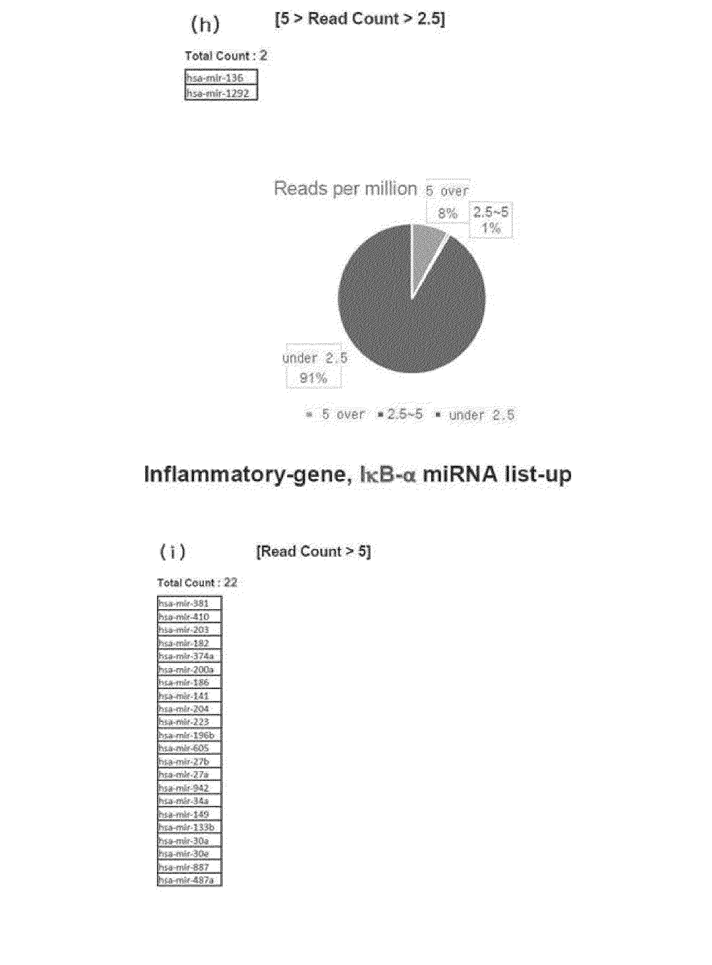

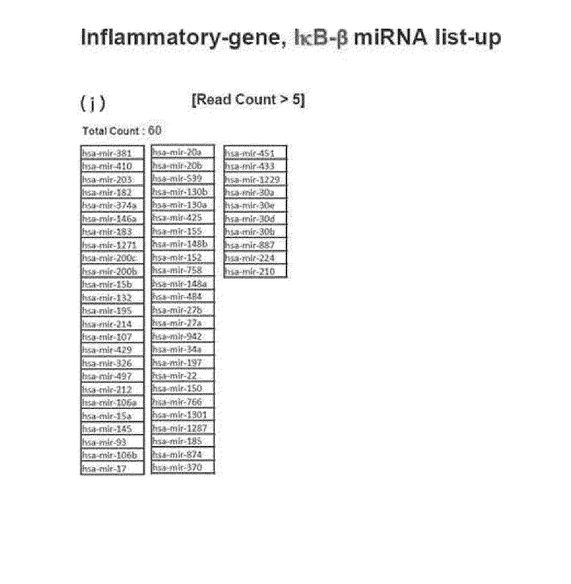

The RNA pellet prepared in Experimental Example 2 was used, and the base sequence of RNA was analyzed by sequencing systems. The method of identifying mi-RNA from the base sequences of the analyzed RNA was carried out in the same manner as in Example 1. FIG. 3 shows the results of inflammatory-gene clustering analysis about p65, p50, IKB-α and IKB-β.

### EXAMPLE 3

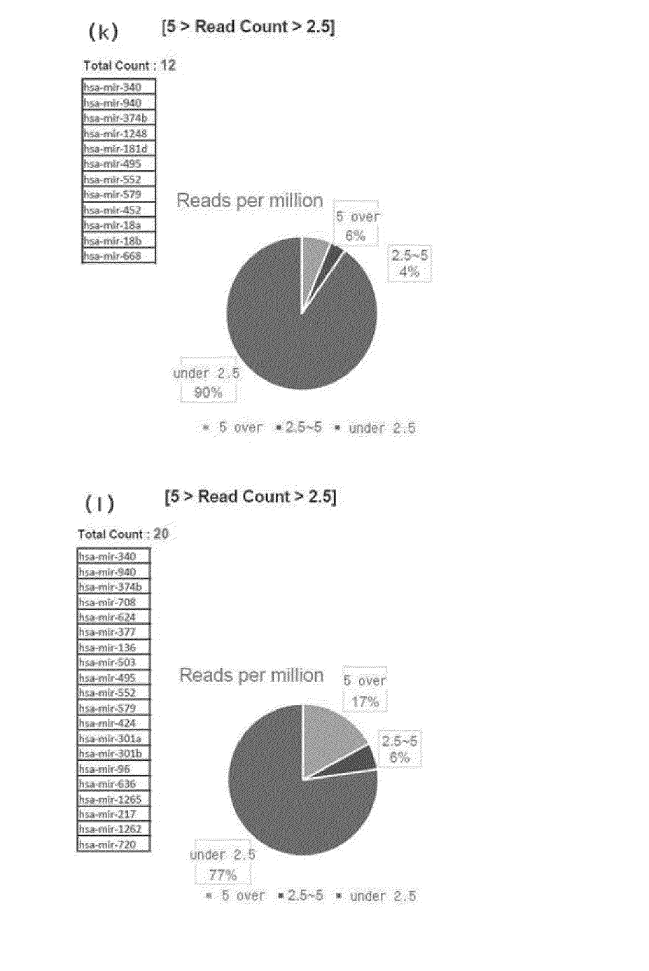

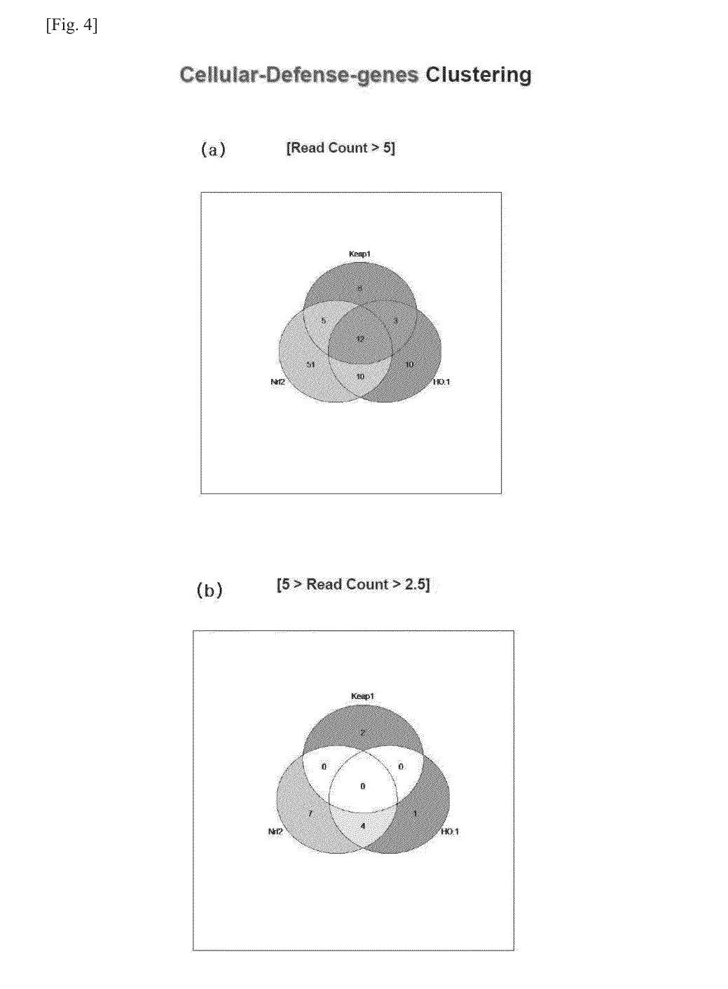

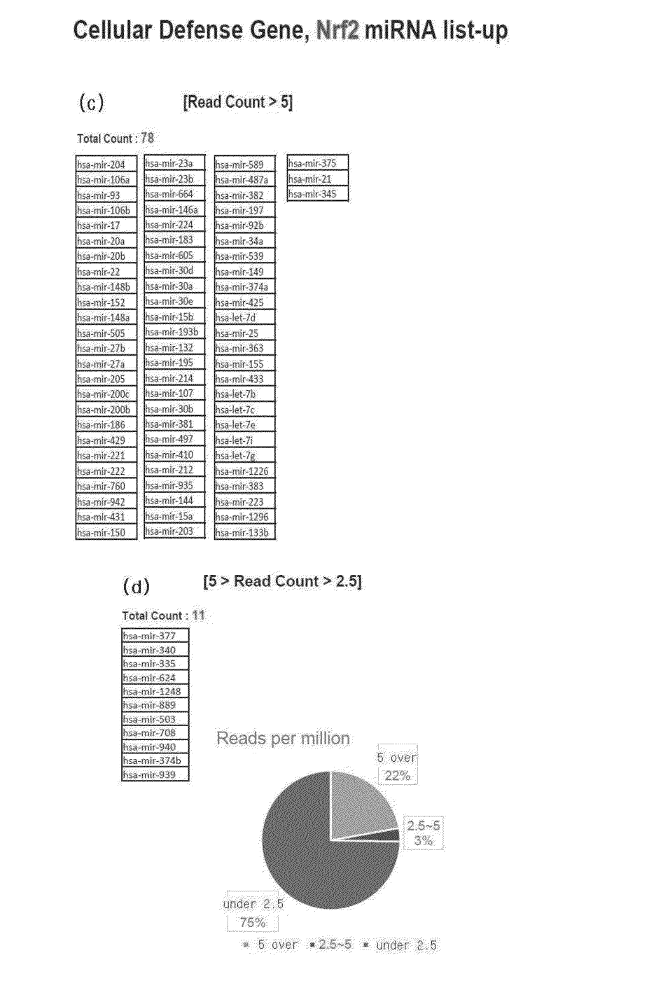

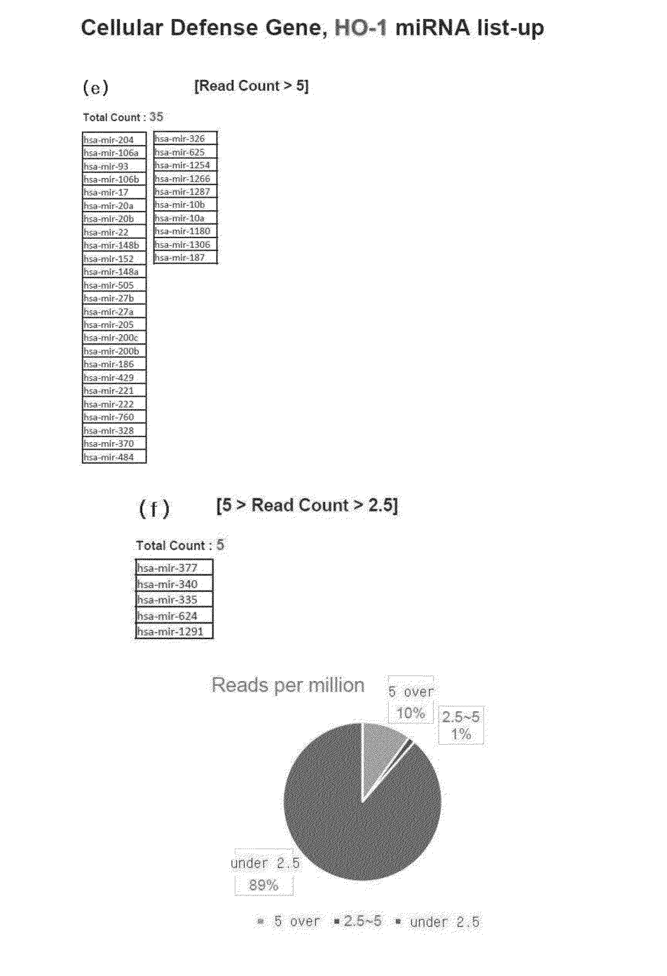

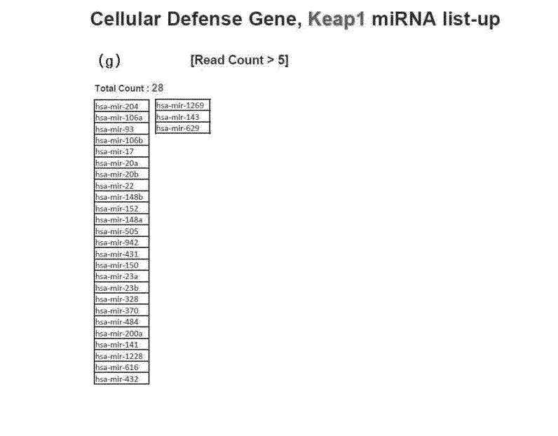

The RNA pellet prepared in Experimental Example 3 was used, and the base sequence of RNA was analyzed by sequencing systems. The method of identifying mi-RNA from the base sequences of the analyzed RNA was carried out in the same manner as in Example 1. FIG. 4 shows the results of cellular defense genes clustering analysis about Keap-1, Nrf-2, and HO-1.

Although the invention has been set forth in detail, one skilled in the art will recognize that numerous changes and modifications can be made, and that such changes and modifications may be made without departing from the spirit and scope of the invention. The patents, patent applications and publications cited in the specification are hereby incorporated by reference herein in their entirety for all purposes.

What is claimed is:

1. An analysis method for mi-RNA ID which comprises:
(a) step of preparing an unknown biological sample;
(b) step of extracting the unknown mi-RNAs from the above biological sample;
(c) step of obtaining common results from the mi-RNA extracted in the step (b) and a reference mi-RNA database;
(d) step of compensating the amount of mi-RNA obtained in the step (c) by the normalization for the comparison of mi-RNA results;
(e) step of performing the primary analysis of the mi-RNA results compensated by the normalization in the step (d), whose read count is more than 5, and the secondary analysis of the same results whose read count is between 2.5 and 5, by comparing to another database other than the above reference mi-RNA database;
(f) step of obtaining the mi-RNA ID from the results that are commonly derived from the results analyzed in the step (e).

2. The analysis method for mi-RNA ID according to claim 1, in which the unknown biological sample in the step (a) is derived from a fresh or frozen colon cancer tissue, cell, saliva, blood, serum or plasma.

3. The analysis method for mi-RNA ID according to claim 1, in which the extraction of the RNA in the step (B) is done by using the Trizol or Triton X-100.

4. The analysis method for mi-RNA ID according to claim 1, in which the reference mi-RNA databases of the step (c) are used miRBase, ASRP, micro RNAMAP, miRGen, CoGemiR, and or miRZipTM.

5. The analysis method for mi-RNA ID according to claim 4, in which one or more of the above reference mi-RNA databases is used more than one database.

6. The analysis method for mi-RNA ID according to claim 1, in which another database in the step (e) are used miRWalK, miRanda, miRDB, RNA-22, Targetscan, TarBase, miRecords, MiRscan, ProMiR, miRDeep, miRanalyzer, Pic Tar, DIANA-microT, RNAhybrid, Mir Target2.

7. The analysis method for micro RNAmi-RNA ID according to claim 1, in which one or more of the above another mi-RNA databases is used.

8. The analysis method for mi-RNA ID according to claim 1, in which the another mi-RNA database in the step (e) gives the read count more than 5 in the primary analysis, and secondly give the read count between 2.5 and 5 in the secondary analysis.

9. The analysis method for mi-RNA ID according to claim 1, in which any one of tumor genes and tumor suppressor genes, genes related to inflammation, inflammation related transcription factors, intracellular antioxidant defense-related genes, intracellular antioxidant defense-related transcription factor are identified by the analysis of mi-RNA ID obtained in the step (f).

10. The analysis method for mi-RNA ID according to claim 9, in which oncogenes and tumor suppressor genes are K-ras, TGF-β, TGF-BR2, Smads4, PTEN, PI3K, EGFR, VEGF, MYC, p53, APC, FOXO1m, Braf and Sirt-1.

11. The analysis method for mi-RNA ID according to claim 9, in which the inflammatory genes are COX-2, and the inflammation-related transcription factor p65, p50, IKB-α and IKB-β.

12. The analysis method for mi-RNA ID according to claim 9, in which the antioxidant defense genes in the cells is HO-1, and the transcription factor related to intracellular antioxidant defenses are Nrf-2 and Keap-1.

13. Biomarkers of colon cancer obtained by the mi-RNA ID analysis result gained through the analysis of the mi-RNA ID according to claim 1.
